SEQUENCE LISTING (35 sequences)

SEQ ID NO: 1 - DNA, 34 bp, Artificial Sequence
  Description of Artificial Sequence Synthetic primer
ataacttcgtatagcatacattatacgaagttat

SEQ ID NO: 2 - DNA, 21 bp, Artificial Sequence
  Description of Artificial Sequence Synthetic primer
aagtgtaagtgcttgaaatgc

SEQ ID NO: 3 - DNA, 21 bp, Artificial Sequence
  Description of Artificial Sequence Synthetic primer
gcatcaaattgagttttgctc

SEQ ID NO: 4 - DNA, 12 bp, Mus musculus
ttcctcgtagaa

SEQ ID NO: 5 - DNA, 11 bp, Mus musculus
ttctcgtagaa

SEQ ID NO: 6 - DNA, 14 bp, Mus musculus
ttctactcgtagaa

SEQ ID NO: 7 - DNA, 13 bp, Mus musculus
ttctaccgtagaa

SEQ ID NO: 8 - DNA, 15 bp, Mus musculus
ttctacctcgtagaa

SEQ ID NO: 9 - DNA, 12 bp, Mus musculus
tcctacgtagaa

SEQ ID NO: 10 - DNA, 12 bp, Mus musculus
ttctacgtagaa

SEQ ID NO: 11 - DNA, 14 bp, Mus musculus
ttctacgagtagaa

SEQ ID NO: 12 - DNA, 15 bp, Mus musculus
ttctacgaggtagaa

SEQ ID NO: 13 - DNA, 16 bp, Mus musculus
ttctacgagggtagaa

SEQ ID NO: 14 - DNA, 17 bp, Mus musculus
ttctacgaggcgtagaa

SEQ ID NO: 15 - DNA, 16 bp, Mus musculus
tcctacgagaatagaa

SEQ ID NO: 16 - DNA, 16 bp, Mus musculus
tcctacgagaatagaa

SEQ ID NO: 17 - DNA, 17 bp, Mus musculus
ttctacgagaggtagaa

SEQ ID NO: 18 - DNA, 17 bp, Mus musculus
ttctacgaggggtagaa

SEQ ID NO: 19 - DNA, 17 bp, Mus musculus
ttctacgaggggtagaa

SEQ ID NO: 20 - DNA, 20 bp, Mus musculus
ttctacgagttctcgtagaa

SEQ ID NO: 21 - DNA, 18 bp, Mus musculus
ttctacgagactgtagaa

SEQ ID NO: 22 - DNA, 20 bp, Mus musculus
ttctacgagttggggtagaa

SEQ ID NO: 23 - DNA, 8 bp, Mus musculus
tcgtagaa

SEQ ID NO: 24 - DNA, 8 bp, Mus musculus
tcgtagaa

SEQ ID NO: 25 - DNA, 11 bp, Mus musculus
ttctcgtagaa

SEQ ID NO: 26 - DNA, 11 bp, Mus musculus
ttctcgtagaa

SEQ ID NO: 27 - DNA, 11 bp, Mus musculus
ttctcgtagaa

SEQ ID NO: 28 - DNA, 11 bp, Mus musculus
ttctcgtagaa

SEQ ID NO: 29 - DNA, 11 bp, Mus musculus
ttctcggagaa

SEQ ID NO: 30 - DNA, 7 bp, Mus musculus
ttctaaa

SEQ ID NO: 31 - DNA, 11 bp, Mus musculus
ttctactagaa

SEQ ID NO: 32 - DNA, 8 bp, Mus musculus
ttctacaa

SEQ ID NO: 33 - DNA, 12 bp, Mus musculus
ttctaggtagaa

SEQ ID NO: 34 - DNA, 12 bp, Mus musculus
ttctaggaggaa

SEQ ID NO: 35 - DNA, 12 bp, Mus musculus
ttctagtaggaa

(19) United States
(12) Patent Application Publication
(10) Pub. No.: US 2011/0185442 A1
(43) Pub. Date: Jul. 28, 2011

(54) Method of generating transgenic organisms using transposons

(75) Inventors: Charalambos Savakis, Crete (GR); Frank Grosveld, Rotterdam (NL)

(73) Assignees: Minos Biosystems Limited; Erasmus University Medical Center Rotterdam

(21) Appl. No.: 12/655,114

(22) Filed: Dec. 23, 2009

Related U.S. Application Data
(63) Continuation of application No. 11/809,579, filed on May 31, 2007, now abandoned.
Application No. 11/809,579 is a division of application No. 10/245,441, filed on Sep. 17, 2002, now abandoned.
Application No. 10/245,441 is a continuation-in-part of application No. PCT/EP01/03341, filed on Mar. 21, 2001.
(60) Provisional application No. 60/195,678, filed on Apr. 7, 2000.

(30) Foreign Application Priority Data
Mar. 21, 2000 (GB) GB00/06753.8

(51) Int. Cl. A01K 67/02

(52) U.S. Cl. 800/22

(57) ABSTRACT

The invention relates to a method for generating a transgenic organism. The invention also relates to a method for detecting and characterizing a genetic mutation in a transgenic organism. The invention further relates to a method for isolating a gene which is correlated with a phenotypic characteristic in a transgenic animal. The invention further relates to a method for isolating an exon in a transgenic animal. The invention also relates to a method for modulating the expression of a gene in an organism.

This application is a continuation application of U.S. Ser. No. 11/809,579 filed on May 31, 2007, which is a divisional application of U.S. continuation-in-part patent application Ser. No. 10/245,441 filed Sep. 17, 2002, which claims priority to PCT No. PCT/EP01/03341, filed Mar. 21, 2001, and also claims the benefit of U.S. Patent Application No. 60/195,678, filed Apr. 7, 2000, and UK Application No. GB00/06753.8, filed Mar. 21, 2000. The entireties of all of these applications are hereby incorporated by reference herein.

The present invention relates to transgenic organisms, and methods for producing such organisms. In particular, the invention relates to transgenic organisms which comprise one or more insertions of a transposable element, or transposon. The transposon is preferably the Minos transposon.

Transposons are genetic elements which are capable of “jumping” or transposing from one position to another within the genome of a species. Transposons are widely distributed amongst animals, including insects.

The availability of genetic methodologies for functional genomic analysis is crucial for the study of gene function and genome organization of complex eukaryotes. Of the three “classical” model animals, the fly, the worm and the mouse, efficient transposon based insertion methodologies have been developed for D. melanogaster and for C. elegans. The introduction of P element mediated transgenesis and insertional mutagenesis in Drosophila (Spradling & Rubin, (1982) Science 218:341-347) transformed Drosophila genetics and formed the paradigm for developing equivalent methodologies in other eukaryotes. However, the P element has a very restricted host range, and therefore other elements have been employed in the past decade as vectors for gene transfer and/or mutagenesis in a variety of complex eukaryotes, including nematodes, plants, fish and a bird.

Minos is a transposable element derived from Drosophila (Franz and Savakis, (1991) 25 NAR 19:6646). It is described in U.S. Pat. No. 5,840,865, which is incorporated herein by reference in its entirety. The use of Minos to transform insects is described in the foregoing US patent.

Mariner is a transposon originally isolated from Drosophila, but since discovered in 30 several invertebrate and vertebrate species. The use of mariner to transform organisms is described in International patent application W099/09817.

Hermes is derived from the common housefly. Its use in creating transgenic insects is described in U.S. Pat. No. 5,614,398, incorporated herein by reference in its entirety.

PiggyBac is a transposon derived from the baculovirus host Trichplusia ni. Its use for germ-line transformation of Medfly has been described by Handler et al., (1998) PNAS (USA) 95:7520-5.

European Patent Application 0955364 (Savakis et al., the disclosure of which is incorporated herein by reference) describes the use of Minos to transform cells, plants and animals. The generation of transgenic mice comprising one or more Minos insertions is described.

International Patent Application W099/07871 describes the use of the Tc1 transposon from C. elegans for the transformation of C. elegans and a human cell line.

The use of Drosophila P elements in D. melanogaster for enhancer trapping and gene tagging has been described; see Wilson et al., (1989) Genes dev. 3:1301; Spradling et al., (1999) Genetics 153:135.

In the techniques described in the prior art, the use of the cognate transposase for inducing transposon jumping is acknowledged to be necessary. Transgenic animals, where described, have the transposase provided in cis or trans, for example by cotransformation with transposase genes.

### SUMMARY OF THE INVENTION

We have now developed an improved protocol for the generation of transgenic animals using transposable elements as a genetic manipulation tool. In the improved protocol, the transposase function is provided by crossing of transgenic organisms in order to produce organisms containing both transposon and transposase in the required cells or tissues. The invention allows tissue-specific, regulatable transposition events to be used for genetic manipulation of organisms.

According to a first aspect of the invention, there is provided a method for generating a transgenic organism, comprising the steps of:
(a) providing a first transgenic organism, which organism comprises, within at least a portion of its tissues or cells, one or more copies of a transposon;
(b) providing a second organism, which organism comprises, in the genome of at least a portion of its tissues or cells, a transposase or one or more copies of a gene encoding a transposase; and
(c) crossing the organism so as to obtain transgenic progeny which comprise, in at least a portion of their tissues or cells, both the transposon and the transposase.

The invention comprises the crossing of two transgenic organisms, wherein one organism comprises, preferably as a result of transgenesis, one or more copies of a transposon; and the other organism comprises, preferably as a result of transgenesis, one or more copies of the cognate transposase. Any organism comprising heterologous or artificially rearranged genetic material is transgenic; it is preferred that a transgenic organism according to the invention is a eukaryotic organism.

As used herein, the term “transposon” refers to a genetic element that can “jump” or tranpose from one position to another within the genome of an organism. In order to be mobilized, a transposon requires intact inverted terminal repeat sequences and the presence of an active transposase. The inverted terminal repeat structures function in the recognition, excision and re-insertion of transposon sequences by a transposase. Transposases are generally encoded by the transposon sequences, but can also be supplied in trans. It is preferred herein that the transposase enzyme required for transposition is not encoded by the transposon sequence itself and is supplied in trans.

It is highly preferred that the transposon is Minos; and/or that the organism is a mammal.

As used herein, the term “transposase” refers to an enzyme that performs the excision and/or insertion activities necessary for the transposition of a transposon. A “cognate” transposase, as referred to herein, is any transposase which is effective to activate transposition of a given transposon, including excision of the transposon from a first integration site and/or integration of the transposon at a second integration site. Preferably, the cognate transposase is the transposase which is naturally associated with the transposon in its in vivo situation in nature. However, the invention also encompasses modified transposases, which may have advantageously improved activities within the scope of the invention.

The transposon may be a natural transposon. Preferably, it is a type-2 transposon, such as Minos. Most, advantageously, it is Minos. Alternative transposons include, but are not limited to mariner, Hermes and piggyBac, the sequences of which are known in the art (see, e.g., U.S. Pat. No. 5,840,865 (Minos), WO 99/09817 (mariner), U.S. Pat. No. 5,614,398 (Hermes) and Handler et al., 1998, Proc. Natl. Acad. Sci. U.S.A. 95: 7520-7525, each of which is incorporated herein by reference). As used herein, a transposon is a specific type of transposon, e.g., “a Minos transposon” if the transposon retains at least the sequences necessary for the excision and/or re-insertion by the cognate transposase enzyme as that term is defined herein.

The invention moreover relates to the use of modified transposons, the modification being the removal or disruption of transposase sequences or the incorporation of one or more heterologous coding sequences and/or expression control sequences. Such coding sequences can include selectable and/or unselectable marker genes, which permit the identification of transposons in the genome and cloning of the loci into which the transposons have been integrated. Suitable markers include fluorescent and/or luminescent polypeptides, such as GFP and derivatives thereof, luciferase, β-galactosidase, or chloramphenicol acetyl transferase (CAT).

As used herein, the term “heterologous” refers to genetic sequences that are from a species other than the organism or transposon of interest. As used herein, the term “homologous” refers to a genetic sequence that is normally carried by the organism or transposon of interest.

As used herein, the term “portion,” when used in reference to the tissues or cells of an organism, means at least one cell of the organism, up to and including all cells of the organism.

As used herein, the term “control sequences” refers to those nucleic acid sequences that mediate the transcription and/or translation of a given nucleic acid sequence. Control sequences include, for example, promoters (both basal and regulated, including, for example, tissue-specific or temporally-regulated promoters, or inducible promoters), enhancers, silencers and locus control regions.

As used herein in regard to the regulation of expression, a “signal” refers to a tissue-specific signal, a developmental signal, or an exogenous signal.

As used herein, the term “inducible expression system” refers to control sequences that permit the variable regulation of expression of an operably linked nucleic acid sequence by the manipulation of one or more parameters, including, for example, the presence, absence or relative amount of a drug.

As used herein, the term “tissue specific signals” refers to those biological signals that mediate the expression of a gene in a manner such that the gene is differentially expressed in at least one tissue of an organism, relative to other tissues of that organism. By “differentially expressed” is meant at least a statistically significant difference (p<0.05) in expression rate or steady state accumulation of the gene product in one tissue, relative to another. Biological signals include, for example the presence, absence, or regulating activity of agents or factors (intracellular or extracellular) involved in, for example, signal transduction, transcription, translation and RNA or protein processing, transport and stability.

The following is a non-exclusive list of tissue specific promoters and literature references containing the necessary sequences to achieve expression characteristic of those promoters in their respective tissues; the entire content of each of these literature references is incorporated herein by reference: Bowman et al., 1995 Proc. Natl. Acad. Sci. USA 92,12115-12119 describe a brain-specific transferrin promoter; the synapsin I promoter is neuron specific (Schoch et al., 1996 J. Biol. Chem. 271, 3317-3323); the necdin promoter is post-mitotic neuron specific (Uetsuki et al., 1996 J. Biol. Chem. 271, 918-924); the neurofilament light promoter is neuron specific (Charron et al., 1995′J. Biol. Chem. 270, 30604-30610); the acetylcholine receptor promoter is neuron specific (Wood et al., 1995 J. Biol. Chem. 270, 30933-30940); the potassium channel promoter is high-frequency firing neuron specific (Gan et al., 1996 J. Biol. Chem. 271, 5859-5865); the chromogranin A promoter is neuroendocrine cell specific (Wu et al., 1995 A. J. Clin. Invest. 96, 568-578); the Von Willebrand factor promoter is brain endothelium specific (Aird et al., 1995 Proc. Natl. Acad. Sci. USA 92, 4567-4571); the flt-1 promoter is endothelium specific (Morishita et al., 1995 J. Biol. Chem. 270, 27948-27953); the preproendothelin-1 promoter is endothelium, epithelium and muscle specific (Harats et al., 1995 J. Clin. Invest. 95, 1335-1344); the GLUT4 promoter is skeletal muscle specific (Olson and Pessin, 1995 J. Biol. Chem. 270, 23491-23495); the Slow/fast troponins promoter is slow/fast twitch myofibre specific (Corin et al., 1995 Proc. Natl. Acad. Sci. USA 92, 6185-6189); the α-Actin promoter is smooth muscle specific (Shimizu et al., 1995 J. Biol. Chem. 270, 7631-7643); the Myosin heavy chain promoter is smooth muscle specific (Kallmeier et al., 1995 J. Biol. Chem. 270, 30949-30957); the E-cadherin promoter is epithelium specific (Hennig et al., 1996 J. Biol. Chem. 271, 595-602); the cytokeratins promoter is keratinocyte specific (Alexander et al., 1995 B. Hum. Mol. Genet. 4, 993-999); the transglutaminase 3 promoter is keratinocyte specific (J. Lee et al., 1996 J. Biol. Chem. 271, 4561-4568); the bullous pemphigoid antigen promoter is basal keratinocyte specific (Tamai et al., 1995 J. Biol. Chem. 270, 7609-7614); the keratin 6 promoter is proliferating epidermis specific (Ramirez et al., 1995 Proc. Natl. Acad. Sci. USA 92, 4783-4787); the collagen α1 promoter is hepatic stellate cell and skin/tendon fibroblast specific (Houglum et al., 1995 J. Clin. Invest. 96, 2269-2276); the type X collagen promoter is hypertrophic chondrocyte specific (Long & Linsenmayer, 1995 Hum. Gene Ther. 6, 419-428); the Factor VII promoter is liver specific (Greenberg et al., 1995 Proc. Natl. Acad. Sci. USA 92, 12347-1235); the fatty acid synthase promoter is liver and adipose tissue specific (Soncini et al., 1995 J. Biol. Chem. 270, 30339-3034); the carbamoyl phosphate synthetase I promoter is portal vein hepatocyte and small intestine specific (Christoffels et al., 1995 J. Biol. Chem. 270, 24932-24940); the Na—K—Cl transporter promoter is kidney (loop of Henle) specific (Igarashi et al., 1996 J. Biol. Chem. 271, 9666-9674); the scavenger receptor A promoter is macrophages and foam cell specific (Horvai et al., 1995 Proc. Natl. Acad. Sci. USA 92, 5391-5395); the glycoprotein IIb promoter is megakaryocyte and platelet specific (Block & Poncz, 1995 Stem Cells 13, 135-145); the yc chain promoter is hematopoietic cell specific (Markiewicz et al., 1996 J. Biol. Chem. 271, 14849-14855); and the CD11b promoter is mature myeloid cell specific (Dziennis et al., 1995 Blood 85, 319-329).

As used herein, the term “developmental signals” refers to those biological signals that mediate the expression of a gene in a manner such that its expression pattern varies relative to the developmental state of the organism or tissue within an organism. An expression pattern “varies” if the expression of the gene or its RNA or polypeptide product undergoes a statistically significant difference (p<0.05) in expression over the course of development of the organism or tissue. Multiple developmentally-regulated promoters are known for a variety of species, notably in model organisms used for developmental studies, e.g., C. elegans, Drosophila, Xenopus, sea urchin, zebrafish, etc., but also in mammals. Non-limiting examples of developmentally-regulated promoters include those for β-globin, T cell receptors, surfactant protein A (SP-A), alphafetoprotein and albumin, among many others.

As used herein, the term “exogenous signals” refers to signals generated by the administration of an agent to the organism. “Exogenous signals” useful according to the invention generally modulate the expression of a drug-regulatable promoter. A number of suitable drug-regulatable promoters and corresponding regulatory drugs are known (see for example, Miller & Whelan, Hum. Gene Ther. 8, 803-815), and include, for example, promoters regulated by tetracycline (or tetracycline analogs that function to regulate tet-responsive promoters), glucocorticoid steroids, sex hormone steroids, metals (e.g., zinc), lipopolysaccharide (LPS), ecdysone and isopropylthiogalactoside (IPTG).

A tetracycline-responsive expression system was originally described by Gossen & Bujard (1992 Proc. Natl. Acad. Sci. USA 89, 5547-5551). In that system, the presence of tet represses expression of genes linked to the tet-responsive promoter (the so-called “tet-off” system). Subsequently, variants of the tet responsive system were developed in which a mutant form of the tet repressor protein binds to DNA in the presence, but not in the absence, of tetracycline or its analogues, resulting in positive regulation by tetracycline and its analogs (the so-called “tet-on” system; see, e.g., WO 96/01313, which is incorporated herein by reference). Tetracycline analogs can be any one of a number of compounds that are closely related to tetracycline and which bind to the tet repressor with a Kₐ of at least about 10⁶/M.

As used herein, the term “locus control region” or “LCR” refers to a DNA sequence which confers high level expression on a group (two or more) of genes by conferring an open chromatin conformation on the chromosomal region comprising such genes. Locus control regions are often located between the genes regulated by the LCR and generally comprise one or more DNAse hypersensitive regions. Numerous LCRs are known in the art. Representative examples are described as follows. The human β-globin LCR is described in, for example, Grosveld et al., 1987, Cell 51: 975-985, Talbot et al., 1989, Nature 338: 352-355, Levings & Bungert, 2002, Eur. J. Biochem. 269: 1589-1599 (review), and GenBank Accession No. AF064190. As another example, an evolutionarily conserved LCR resides between 3.1 and 3.7 kb upstream of the human red visual pigment gene (Nathans et al., 1989, Science 245: 831-838; Wang et al., 1992, Neuron 9: 429-440). The 3′ IgH LCR is provided in GenBank Accession No. Y14406. The murine tyrosinase LCR is provided in GenBank Accession No. AF364302. The human CD2 gene LCR is described by Kaptein et al., 1998, Gene Ther. 5: 320-330. The murine T cell receptor a/Dad1 LCR is described by Ortiz et al., 2001, J. Immunol. 167: 3836-3845.

In an advantageous embodiment, the transposase may be expressed in the transgenic organisms in a regulatable manner. This means that the activation of the transposon can be determined according to any desired criteria. For example, the transposase may be placed under the control of tissue-specific sequences, such that it is only expressed at desired locations in the transgenic organism. Such sequences may, for example, comprise tissue-specific promoters, enhancers and/or locus control sequences.

Moreover, the transposase may be placed under the control of one or more sequences which confer developmentally-regulated expression. This will result in the transposons being activated at a given stage in the development of the transgenic animal or its progeny.

Using the techniques of the invention, gene modification events can be observed at a very high frequency, due to the efficiency of mobilisation and insertion of transposons. Moreover, the locus of the modification may be identified precisely by locating the transposon insertion. Sequencing of flanking regions allows identification of the locus in databases, potentially without the need to sequence the locus. Moreover, the use of transposons provides a reversible mutagenesis strategy, such that modifications can be reversed in a controlled manner.

As used herein, the term “genetically manipulate” refers to a process that artificially alters the genetic makeup of an organism. The transposon-mediated excision or insertion of a transgene sequence as described herein is one example of genetic manipulation.

The transposon may be inserted into a gene. Preferably, the transposon is inserted into a highly transcribed gene, resulting in the localisation of said transposon in open chromatin. This increases the accessibility of the transposon which may result in increased transposition frequencies.

As used herein, the term “open chromatin” refers to a region of chromatin that is at least 10-fold more sensitive to the action of an endonucleoase, e.g., DNAse I, than surrounding regions. Because opening of the chromatin is a prerequisite to transcription activity, DNAse I sensitivity provides a measure of the transcriptional potentiation of a chromatin region; greater DNAse sensitivity generally corresponds to greater transcription activity. DNAse hypersensitivity assays are described by Weintraub & Groudine, 1976, Science 193: 848-856, incorporated herein by reference. “Highly transcribed” or “highly expressed” regions or genes are regions of open chromatin structure (i.e., at least 10-fold more DNAse I sensitive) that are transcribed and are preferably more than 10-fold more sensitive to DNAse I cleavage, e.g., preferably at least 20-fold or more, preferably 50-fold or 100 fold or more sensitive, than surrounding regions.

Moreover, the transposon may itself comprise, between the transposon ends, a highly-transcribed gene. This will cause activation of the chromatin structure into which the transposon integrates, facilitating access of the transposase thereto.

The transposon may be inserted into the gene by recombination. Furthermore, the transposon may be inserted into the gene by recombination in cells such as ES cells.

According to a second aspect of the invention, there is provided a method for detecting and characterising a genetic mutation in a transgenic organism, comprising the steps of:
(a) generating a transgenic organism by a procedure according to the first aspect of the invention;
(b) characterising the phenotype of the transgenic organism;
(c) detecting the position of one or more transposon insertion events in the genome of the organism; and
(d) correlating the position of the insertion events with the observed phenotype, the position of the insertion events being indicative of the location of one or more gene loci connected with the observed phenotype.

As used herein, the term “reversion of gene disruptions” refers to the restoration of the expression of a polypeptide that was disrupted by the prior insertion of a transposon, which restoration follows the excision of the inserted transposon by a transposase. By “restoration” is meant that, following reversion, the expressed polypeptide is more abundant (i.e., at least 5% more abundant) or has greater activity (i.e., at least 5% greater activity) than prior to the reversion event.

As used herein, the term “characterize the phenotype” refers to the measurement of one or more parameters that determines the phenotype of an organism made transgenic by the transposon-mediated methods disclosed herein, relative to that parameter in a reference organism that is not made transgenic according to the transposon-mediated methods described herein. Non-limiting examples of phenotypic parameters include the measurement of the presence, absence, amount or activity of a polypeptide or one or more products of a reaction requiring or catalyzed by that polypeptide.

As used herein, the term “correlating the position of the insertion events with the observed phenotype” means determining the location of transposon insertion events in transgenic organisms according to the invention that exhibit a particular observed phenotype. Determining the location or detecting the position of an insertion can be performed on the chromosomal level, e.g., by fluorescence in situ hybridization, or, preferably, at the level of determining the sequence of those genomic regions flanking the insertion site. The observed phenotype can be, for example, activation or reversion of expression of a polypeptide or, alternatively, inactivation of the expression of a polypeptide.

The generation of genetic mutations in transgenic organisms as a result of transposon insertion after crossing of transgenic organisms according to the invention gives rise to novel phenotypic variations in the organisms, which can be traced back to insertion events in the genome of the organism. Transposon excisions characteristically result in the insertion of a small number of nucleotides into the host genome, left behind by the transposon and the recombination events associated with its insertion and subsequent excision. Small insertions may have small phenotypic effects, for example resulting from the insertion of a few amino acids into the sequence of a polypeptide. Alternatively, the effects may be more pronounced, possibly including the complete inactivation of a gene.

Transposon insertions are more likely to have significant phenotypic consequences, on the grounds that the insertion is much larger.

If a transposon is inserted into an intron of a gene, resulting in inactivation of the gene, its excision leads, in the majority of cases, to restoration of gene activity. Thus, the invention provides a reversible mutagenesis procedure, in which a gene can be inactivated and subsequently restored.

Insertion events may be detected by screening for the presence of the transposon, by probing for the nucleic acid sequence of the transposon. Excisions may also be identified by the “signature” sequence left behind upon excision.

In a preferred embodiment, transposons may be used to upregulate the expression of genes. For example, a transposon may be modified to include an enhancer or other transcriptional activation element. Mobilisation and insertion of such a transposon in the vicinity of a gene upregulates expression of the gene or gene locus. This embodiment has particular advantage in the isolation of oncogenes, which may be identified in clonal tumours by localisation of the transposon.

According to a third aspect, there is provided a method for isolating a gene which is correlated with a phenotypic characteristic in a transgenic animal, comprising the steps of:
(a) generating a transgenic organism by a procedure according to the first aspect of the invention;
(b) characterising the phenotype of the transgenic organism;
(c) detecting the position of one or more transposon insertion events in the genome of the organism; and
(d) cloning the genetic loci comprising the insertions.

The invention provides clear advantages in functional genomics, since gene disruption or activation by transposon jumping is easily traced due to tagging by the transposon.

According to a fourth aspect, there is provided a method for isolating an enhancer in a transgenic animal, comprising the steps of:
(a) generating a transgenic organism by a procedure according to the first aspect of the invention, wherein the transposon comprises a reporter gene under the control of a minimal promoter such that it is expressed at a basal level;
(b) assessing the level of expression of the reporter gene in one or more tissues of the transgenic organism;
(c) identifying and cloning genetic loci in which the modulation of the reporter gene is increased or decreased compared to the basal expression level; and
(d) characterising the cloned genetic loci.

According to a fifth aspect, there is provided a method for isolating an exon of an endogenous gene in a transgenic animal, comprising the steps of:
(a) generating a transgenic organism by a procedure according to the first aspect of the invention, wherein the transposon comprises a reporter gene which lacks translation initiation sequences but includes splice acceptor sequences;
(b) identifying tissues of the organism in which the reporter gene is expressed; and
(c) cloning the genetic loci comprising the expressed reporter gene.

As used herein, the term “lacks translation initiation sequences” means that the reporter gene does not have an in frame ATG codon within a Kozak consensus sequence (described in Kozak, 1986, Cell 44: 283, and refined in Kozak, 1987, J. Mol. Biol. 196: 947, Kozak, 1987, Nucl. Acids Res. 15: 8125 and Kozak, 1989, J. Cell Biol. 108: 229). A gene that lacks translation initiation sequences will not be expressed unless it is provided with such sequences, e.g., by insertion mutagenesis.

As used herein, the term “includes splice acceptor sequences” means that the reporter gene coding sequence in the transposon is preceded by a branch site consensus sequence (UCPuAPy), 20 to 50 nucleotides 5′ of a 3′ splice acceptor sequence AG/G (where the 3′ G is the splice acceptor).

The invention may be used to provided in vivo enhancer trap and exon trap functions, by inserting transposons which comprise marker genes which are modulated in their expression levels by the proximity with enhancers or exons. Suitable constructs for such applications are described in EP 0955364 and known in the art. Since transposon activation may be effected in a tissue-specific or developmentally regulated manner, the invention permits the trapping of enhancers and/or exons which are subject to similar regulation in the transgenic organism.

As used herein the term “enhancer” refers to a eukaryotic promoter sequence element that increases transcriptional efficiency in a manner that is relatively independent of position and orientation with respect to a nearby gene (see, e.g., Khoury and Gruss, 1983, Cell 33:313-314). The term “relatively independent” as used in the preceding sentence means independent of position and orientation effects relative to basal promoter elements, which generally have strict position and/or orientation requirements for proper promoter function. The ability of enhancer sequences to function upstream from, within or downstream from eukaryotic genes distinguishes them from basal promoter elements.

As used herein, the term “minimal promoter” refers to the minimal expression control element that is capable of initiating transcription of a selected DNA sequence to which it is operably linked. A minimal promoter frequently consists of a TATA box or TATA-like box but can include an initiator element (see, e.g., Smale & Baltimore, 1989, Cell 57: 103) containing a transcriptional initiation site located about 20-50 bases downstream of the TATA box. Generally, no additional upstream elements are present in a minimal promoter. Numerous minimal promoter sequences are known in the art.

As used herein, the term “basal level,” when used in reference to gene expression, means that level of expression that occurs from a minimal promoter.

As used herein, the terms “increased”, “decreased”, or “modulated” mean at least a 5% change in the entity being measured, relative to a reference. For example, reporter gene expression is increased if it is at least 5% higher under a given set of circumstances relative to a different set of circumstances, e.g., the presence, versus the absence of a stimulus.

As used herein, the term “characterizing the cloned genetic loci” refers to determining one or more parameters with regard to the cloned loci, including, for example, nucleic acid sequence, amino acid sequence of open reading frames, or similarity of either of these parameters to that of a known genetic locus.

According to a sixth aspect, there is provided a method for modulating the expression of a gene in an organism, comprising the steps of:
(a) generating a library of transgenic organisms according to the first aspect of the invention; and
(b) selecting from said library one or more transgenic organisms in which the expression of a gene of interest is modulated as a result of one or more transposon insertion events.

As used herein, the term “library” refers to a plurality of transgenic organisms made using a transposon-mediated transgenesis method as disclosed herein. Generally, a library comprises members that while similar in most aspects, differ in one or more other aspects from other members of the library. Thus, a library of transgenic organisms would generally be all of the same species and all contain the same or related transposon or transposase, yet differ in sequences within the transposon sequence from member to member.

The invention moreover comprises transgenic animals suitable for crossing in a method according to the invention, and thus encompasses a transgenic organism comprising one or more copies of a heterologous transposon, said transgenic organism being free of nucleic acid sequences encoding the cognate transposase enzyme, and a transgenic organism encoding a transposase enzyme, said transgenic organism being free of the cognate transposon.

As used herein, the term “free of nucleic acid sequences encoding the cognate transposase enzyme” means that the transgenic organism does not encode a functional cognate transposase enzyme in its genome. A “functional” transposase enzyme is capable of performing excision and/or insertion of its cognate transposon sequence.

As used herein, “free of the cognate transposon” means that the transgenic organism does not encode in its genome a transoposon sequence that can be either excised and/or re-inserted by the cognate transposase.

### DESCRIPTION OF THE FIGURES

FIG. 1. Minos derived vectors. Minos inverted terminal repeats are shown as thick black arrows. White blocks outside these arrows indicate the sequences flanking the original Minos element in the D. hydei genome. Arrowheads indicate the positions of primers used to detect Minos excisions. Small arrows indicate the direction of transcription of the GFP and transposase genes. Black bars represent fragments used as probes.

FIG. 2. Tissue specific expression of Minos transposase in transgenic mice. Northern blot analysis of thymus, spleen and kidney RNA isolated from TM2/+ mice (40-hr exposure). Control RNA is from thymus of a non-transgenic mouse. The lower panel shows the signal obtained upon re-hybridisation of the same filter with a mouse actin probe (3-br exposure).

FIG. 3. Transposase dependent, tissue-specific excision of a Minos transposon in mice. Oligonucleotide primers flanking the transposon were used for PCR and the products were analysed by agarose gel electrophoresis. Left panel: Transposase-dependent excision in the thymus. Template DNA used: Lane 1, non transgenic; lane 2, TM2/+; lanes 3-7, MCG/+; lanes 8-12, MCG/+TM2/+. Right panel: Excision in various tissues of transposase-expressing mice. Template DNA used: Lanes 1, 3, 5, 7, 9, 11, from MCG/+ mice. Lanes 2, 4, 6, 8, 10, 12, from MCG/+TM2/+ mice. Lanes 1-2, thymus. Lanes 3-4, spleen. Lanes 5-6, liver. Lanes 7-8, kidney. Lanes 9-10, brain. Lanes 11-12, muscle. Lane 13, no DNA added.

FIG. 4. Footprints left behind at chromosomal sites after Minos excision. DNA is extracted from thymus and spleen of a double transgenic mouse (top), or from an embryonic fibroblast cell line from a MCG/+ mouse after transfection with a transposase-expressing plasmid (bottom) and used as template for PCR with the flanking primers. PCR-amplified bands were cloned and 32 clones (19 from thymus and spleen and 13 from fibroblast cells) were sequenced. TA is the target site duplication. Nucleotides in red correspond to the ends of the transposon terminal repeats; nucleotides in blue are of unknown origin. The flanking nucleotides and TA repeats are aligned.

FIG. 5. FISH analysis of Minos transpositions in thymus and spleen. Chromosomes were stained with DAPI Panels A and B are from the same MCG/+ metaphase nucleus, probed with a GFP and a telomere 14 specific probe, respectively. Panels C to F are nuclei probed with GFP. Panels C and D are from thymus and spleen respectively from the same MCG/+, TM2/+ mouse. Panels E and F are from spleen of two different MCG/+, TM2/+ mice. Yellow arrowheads indicate the original integration site of the transposon transgene, near the telomere of chromosome 14. Green arrowheads indicate the telomeres of chromosome 14. Red arrowheads indicate transposition events.

### DETAILED DESCRIPTION OF THE INVENTION

Although in general the techniques mentioned herein are well known in the art, reference may be made in particular to Sambrook et al., Molecular Cloning, A Laboratory Manual (1989) and Ausubel et al., Short Protocols in Molecular Biology (1999) 4^{th} Ed, John Wiley & Sons, Inc.

A transgenic organism of the invention is preferably a multicellular eukaryotic organism, such as an animal, a plant or a fungus.

The organism is preferably an animal, more preferably a mammal. Advantageously, the organism is not an insect. Preferably, the organism is not D. melanogaster.

In a preferred embodiment, the organism is a plant.

Animals include animals of the phyla cnidaria, ctenophora, platyhelminthes, nematoda, annelida, mollusca, chelicerata, uniramia, crustacea and chordata. Uniramians include the subphylum hexapoda that includes insects such as the winged insects. Chordates include vertebrate groups such as mammals, birds, reptiles and amphibians. Particular examples of mammals include non-human primates, cats, dogs, ungulates such as cows, goats, pigs, sheep and horses and rodents such as mice, rats, gerbils and hamsters.

Plants include the seed-bearing plants angiosperms and conifers. Angiosperms include dicotyledons and monocotyledons. Examples of dicotyledonous plants include tobacco, (Nicotiana plumbaginifolia and Nicotiana tabacum), arabidopsis (Arabidopsis thaliana), Brassica napus, Brassica nigra, Datura innoxia, Vicia narbonensis, Vicia faba, pea (Pisum sativum), cauliflower, carnation and lentil (Lens culinaris). Examples of monocotyledonous plants include cereals such as wheat, barley, oats and maize.

### Production of Transgenic Animals

Techniques for producing transgenic animals are well known in the art. A useful general textbook on this subject is Houdebine, Transgenic animals—Generation and Use (Harwood Academic, 1997)—an extensive review of the techniques used to generate transgenic animals from fish to mice and cows.

Advances in technologies for embryo micromanipulation now permit introduction of heterologous DNA into, for example, fertilised mammalian ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In a highly preferred method, developing embryos are infected with a retrovirus containing the desired DNA, and transgenic animals produced from the infected embryo. In a most preferred method, however, the appropriate DNAs are coinjected into the pronucleus or cytoplasm of embryos, preferably at the single cell stage, and the embryos allowed to develop into mature transgenic animals. Those techniques are well known. See reviews of standard laboratory procedures for microinjection of heterologous DNAs into mammalian fertilised ova, including Hogan et al., Manipulating the Mouse Embryo, (Cold Spring Harbor Press 1986); Krimpenfort et al., (1991) Bio/Technology 9:844; Palmiter et al., (1985) Cell 41:343; Kraemer et al., Genetic manipulation of the Mammalian Embryo, (Cold Spring Harbor Laboratory Press 1985); Hammer et al., (1985) Nature 315:680; Wagner et al., U.S. Pat. No. 5,175,385; Krimpenfort et al., U.S. Pat. No. 5,175,384, the respective contents of which are incorporated herein by reference.

Another method used to produce a transgenic animal involves microinjecting a nucleic acid into pro-nuclear stage eggs by standard methods. Injected eggs are then cultured before transfer into the oviducts of pseudopregnant recipients.

Transgenic animals may also be produced by nuclear transfer technology as described in Schnieke, A. E. et al., (1997) Science 278:2130 and Cibelli, J. B. et al., (1998) Science 280:1256. Using this method, fibroblasts from donor animals are stably transfected with a plasmid incorporating the coding sequences for a polypeptide of interest under the control of regulatory sequences. Stable transfectants are then fused to enucleated oocytes, cultured and transferred into female recipients.

Analysis of animals which may contain transgenic sequences would typically be performed by either PCR or Southern blot analysis following standard methods.

By way of a specific example for the construction of transgenic mammals, such as cows, nucleotide constructs comprising a sequence encoding a DNA binding molecule are microinjected using, for example, the technique described in U.S. Pat. No. 4,873,191, into oocytes which are obtained from ovaries freshly removed from the mammal. The oocytes are aspirated from the follicles and allowed to settle before fertilisation with thawed frozen sperm capacitated with heparin and prefractionated by Percoll gradient to isolate the motile fraction.

The fertilised oocytes are centrifuged, for example, for eight minutes at 15,000 g to visualise the pronuclei for injection and then cultured from the zygote to morula or blastocyst stage in oviduct tissue-conditioned medium. This medium is prepared by using luminal tissues scraped from oviducts and diluted in culture medium. The zygotes must be placed in the culture medium within two hours following microinjection.

Oestrous is then synchronized in the intended recipient mammals, such as cattle, by 30 administering coprostanol. Oestrous is produced within two days and the embryos are transferred to the recipients 5-7 days after oestrous. Successful transfer can be evaluated in the offspring by Southern blot.

Alternatively, the desired constructs can be introduced into embryonic stem cells (ES cells) and the cells cultured to ensure modification by the transgene. The modified cells are then injected into the blastula embryonic stage and the blastulas replaced into pseudopregnant hosts. The resulting offspring are chimeric with respect to the ES and host cells, and nonchimeric strains which exclusively comprise the ES progeny can be obtained using conventional cross-breeding. This technique is described, for example, in WO91/10741.

### Production of Transgenic Plants

Techniques for producing transgenic plants are well known in the art. Typically, either whole plants, cells or protoplasts may be transformed with a suitable nucleic acid construct encoding a DNA binding molecule or target DNA (see above for examples of nucleic acid constructs). There are many methods for introducing transforming DNA constructs into cells, but not all are suitable for delivering DNA to plant cells. Suitable methods include Agrobacterium infection (see, among others, Turpen et al., (1993) J. Virol. Methods 42:227-239) or direct delivery of DNA such as, for example, by PEG-mediated transformation, by electroporation or by acceleration of DNA coated particles. Acceleration methods are generally preferred and include, for example, microprojectile bombardment. A typical protocol for producing transgenic plants (in particular monocotyledons), taken from U.S. Pat. No. 5,874,265, is described below.

An example of a method for delivering transforming DNA segments to plant cells is microprojectile bombardment. In this method, non-biological particles may be coated with nucleic acids and delivered into cells by a propelling force. Exemplary particles include those comprised of tungsten, gold, platinum, and the like.

A particular advantage of microprojectile bombardment, in addition to it being an effective means of reproducibly stably transforming both dicotyledons and monocotyledons, is that neither the isolation of protoplasts nor the susceptibility to Agrobacterium infection is required. An illustrative embodiment of a method for delivering DNA into plant cells by acceleration is a Biolistics Particle Delivery System, which can be used to propel particles coated with DNA through a screen, such as a stainless steel or Nytex screen, onto a filter surface covered with plant cells cultured in suspension. The screen disperses the tungsten-DNA particles so that they are not delivered to the recipient cells in large aggregates. It is believed that without a screen intervening between the projectile apparatus and the cells to be bombarded, the projectiles aggregate and may be too large for attaining a high frequency of transformation. This may be due to damage inflicted on the recipient cells by projectiles that are too large.

For the bombardment, cells in suspension are preferably concentrated on filters. Filters containing the cells to be bombarded are positioned at an appropriate distance below the microprojectile stopping plate. If desired, one or more screens are also positioned between the gun and the cells to be bombarded. Through the use of techniques set forth herein one may obtain up to 1000 or more clusters of cells transiently expressing a marker gene (“foci”) on the bombarded filter. The number of cells in a focus which express the exogenous gene product 48 hours post-bombardment often range from 1 to 10 and average 2 to 3.

After effecting delivery of exogenous DNA to recipient cells by any of the methods discussed above, a preferred step is to identify the transformed cells for further culturing and plant regeneration. This step may include assaying cultures directly for a screenable trait or by exposing the bombarded cultures to a selective agent or agents.

An example of a screenable marker trait is the red pigment produced, under the control of the R-locus in maize. This pigment may be detected by culturing cells on a solid support containing nutrient media capable of supporting growth at this stage, incubating the cells at, e.g., 18° C. and greater than 180 μE m⁻² s⁻¹, and selecting cells from colonies (visible aggregates of cells) that are pigmented. These cells may be cultured further, either in suspension or on solid media.

An exemplary embodiment of methods for identifying transformed cells involves 30 exposing the bombarded cultures to a selective agent, such as a metabolic inhibitor, an antibiotic, herbicide or the like. Cells which have been transformed and have stably integrated a marker gene conferring resistance to the selective agent used, will grow and divide in culture. Sensitive cells will not be amenable to further culturing.

To use the bar-bialaphos selective system, bombarded cells on filters are resuspended in nonselective liquid medium, cultured (e.g. for one to two weeks) and transferred to filters overlaying solid medium containing from 1-3 mg/l bialaphos. While ranges of 1-3 mg/l will typically be preferred, it is proposed that ranges of 0.1-50 mg/l will find utility in the practice of the invention. The type of filter for use in bombardment is not believed to be particularly crucial, and can comprise any solid, porous, inert support.

Cells that survive the exposure to the selective agent may be cultured in media that supports regeneration of plants. Tissue is maintained on a basic media with hormones for about 2-4 weeks, then transferred to media with no hormones. After 2-4 weeks, shoot development will signal the time to transfer to another media.

Regeneration typically requires a progression of media whose composition has been 15 modified to provide the appropriate nutrients and hormonal signals during sequential developmental stages from the transformed callus to the more mature plant. Developing plantlets are transferred to soil, and hardened, e.g., in an environmentally controlled chamber at about 85% relative humidity, 600 ppm CO₂, and 250 μE m⁻² s⁻¹ of light. Plants are preferably matured either in a growth chamber or greenhouse. Regeneration will typically take about 3-12 weeks. During regeneration, cells are grown on solid media in tissue culture vessels. An illustrative embodiment of such a vessel is a petri dish. Regenerating plants are preferably grown at about 19° C. to 28° C. After the regenerating plants have reached the stage of shoot and root development, they may be transferred to a greenhouse for further growth and testing.

Genomic DNA may be isolated from callus cell lines and plants to determine the presence of the exogenous gene through the use of techniques well known to those skilled in the art such as PCR and/or Southern blotting.

Several techniques exist for inserting the genetic information, the two main principles being direct introduction of the genetic information and introduction of the genetic information by use of a vector system. A review of the general techniques may be found in articles by Potrykus, (Annu. Rev. Plant Physiol. Plant Mol. Biol. [1991] 42:205-225) and Christou, (Agro-Food-Industry Hi-Tech March/April 1994 17-27).

The vector system used may comprise one vector, but it can comprise at least two vectors. In the case of two vectors, the vector system is normally referred to as a binary vector system. Binary vector systems are described in further detail in Gynheung An et al., (1980) Binary Vectors, Plant Molecular Biology Manual A3, 1-19.

One extensively employed system for transformation of plant cells with a given promoter or nucleotide sequence or construct is based on the use of a Ti plasmid from Agrobacterium tumefaciens or a Ri plasmid from Agrobacterium rhizogenes (An et al., (1986) Plant Physiol. 81:301-305 and Butcher D. N. et al., (1980) Tissue Culture Methods for Plant Pathologists, eds.: D. S. Ingrains and J. P. Helgeson, 203-208).

Several different Ti and Ri plasmids have been constructed which are suitable for the construction of the plant or plant cell constructs described above.

### Transposons

Minos transposons, and their cognate transposase, are described in detail in U.S. Pat. No. 5,840,865 and European patent application EP 0955364, the disclosures of which are incorporated herein by reference. Minos transposons may be modified, for instance to insert one or more selectable marker genes for example as referred to herein, according to general techniques. Specific techniques for modifying Minos are set forth in EP 0955364.

### Marker Genes

Preferred marker genes include genes which encode fluorescent polypeptides. For 30 example, green fluorescent proteins (“GFPs”) of cnidarians, which act as their energy-transfer acceptors in bioluminescence, can be used in the invention. A green fluorescent protein, as used herein, is a protein that fluoresces green light, and a blue fluorescent protein is a protein that fluoresces blue light. GFPs have been isolated from the Pacific Northwest jellyfish, Aequorea victoria, from the sea pansy, Renilla reniformis, and from Phialidium gregarium. (Ward et al., (1982) Photochem. Photobiol., 35:803-808; Levine et al., (1982) Comp. Biochem. Physiol., 72B:77-85). See also Matz, et al., 1999, ibid for fluorescent proteins isolated recently from Anthoza species (accession nos. AF168419, AF168420, AF168421, AF168422, AF168423 and AF168424).

A variety of Aequorea-related GFPs having useful excitation and emission spectra have been engineered by modifying the amino acid sequence of a naturally occurring GFP from Aequorea victoria (Prasher et al., (1992) Gene 111:229-233; Heim et al., (1994) Proc. Natl. Acad. Sci. U.S.A., 91:12501-12504; PCT/US95/14692). As used herein, a fluorescent protein is an Aequorea-related fluorescent protein if any contiguous sequence of 150 amino acids of the fluorescent protein has at least 85% sequence identity with an amino acid sequence, either contiguous or non-contiguous, from the wild-type Aequorea green fluorescent protein (SwissProt Accession No. P42212). More preferably, a fluorescent protein is an Aequorea-related fluorescent protein if any contiguous sequence of 200 amino acids of the fluorescent protein has at least 95% sequence identity with an amino acid sequence, either contiguous or non-contiguous, from the wild type Aequorea green fluorescent protein of SwissProt Accession No. P42212. Similarly, the fluorescent protein may be related to Renilla or Phialidium wild-type fluorescent proteins using the same standards.

Aequorea-related fluorescent proteins include, for example, wild-type (native) Aequorea victoria GFP, whose nucleotide and deduced amino acid sequences are presented in Genbank Accession Nos. L29345, M62654, M62653 and others Aequorea-related engineered versions of Green Fluorescent Protein, of which some are listed above. Several of these, i.e. P4, P4-3, W7 and W2, fluoresce at a distinctly shorter wavelength than wild type.

### Identification of Insertion and Excision Events

Minos transposons, and sites from which transposons have been excised, may be identified by sequence analysis. Minos typically integrates at a TA base pair, and on excision leaves behind a duplication of the target TA sequence, flanking the four terminal nucleotides of the transposon. The presence of this sequence, or related sequences, may be detected by techniques such as sequencing, PCR and/or hybridisation.

Inserted transposons may be identified by similar techniques, for example using PCR primers complementary to the terminal repeat sequences.

### Regulation of Transposase Expression

Coding sequences encoding the transposase may be operatively linked to regulatory sequences which modulate transposase expression as desired. Control sequences operably linked to sequences encoding the transposase include promoters/enhancers and other expression regulation signals. These control sequences may be selected to be compatible with the host organism in which the expression of the transposase is required. The term promoter is well-known in the art and encompasses nucleic acid regions ranging in size and complexity from minimal promoters to promoters including upstream elements and enhancers.

The promoter is typically selected from promoters which are functional in cell types homologous to the organism in question, or the genus, family, order, kingdom or other classification to which that organism belongs, although heterologous promoters may function—e.g. some prokaryotic promoters are functional in eukaryotic cells. The promoter may be derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of α-actin, β-actin, tubulin) or, alternatively, a tissue-specific manner (such as promoters of the genes for pyruvate kinase). They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It is moreover advantageous for the promoters to be inducible so that the levels of expression of the transposase can be regulated. Inducible means that the levels of expression obtained using the promoter can be regulated. A widely used system of this kind in mammalian cells is the tetO promoter-operator, combined with the tetracycline/doxycycline-repressible transcriptional activator tTA, also called Tet-Off gene expression system (Gossen, M. & Bujard, H., (1992) Tight control of gene expression in mammalian cells by tetracycline responsive promoters, Proc. Natl. Acad. Sci. U.S.A. 89:5547-5551), or the doxycycline-inducible rtTA transcriptional activator, also called Tet-On system (Gossen, M., Freundlieb, S., Bender, G., Muller, G., Hillen, W. & Bujard, H., (1995) Transcriptional activation by tetracycline in mammalian cells, Science 268:1766-1769).

In the Tet-Off system, gene expression is turned on when tetracycline (Tc) or doxycycline (Dox; a Tc derivative) is removed from the culture medium. In contrast, expression is turned on in the Tet-On system by the addition of Dox. Procedures for establishing cell lines carrying the transcriptional activator gene and the Tet-regulatable gene stably integrated in its chromosomes have been described. For example see http://www.clontech.com/techinfo/manuals/PDF/PT3001-1.pdf. For example, the Tet-On system may be employed for tetracycline-inducible expression of Minos transposase in a transgenic animal. A doubly transgenic animal is generated by standard homologous recombination ES cell technology. Two constructs are used: first, a construct containing the rtTA gene under a constitutive promoter. An example of such construct is the pTet-On plasmid (Clontech) which contains the gene encoding the rtTA activator under control of the Cytomegalovirus immediate early (CMV) promoter. The rtTA transcriptional activator encoded by this construct is active only in the presence of Doxycycline. The second construct contains the Minos transposase gene under control of the tetracycline-response element, or TRE. The TRE consists of seven direct repeats of a 42-bp sequence containing the tet operator (tetO), and is located just upstream of the minimal CMV promoter, which lacks the enhancer elements normally associated with the CMV immediate early promoter. Because these enhancer elements are missing, there is no “leaky” expression of transposase from the TRE in the absence of binding by rtTA. An example of such construct is the pTRE2 plasmid (Clontech) in the MCS of which is inserted the gene encoding Minos transposase. In cells stably transformed with the two constructs, rtTA is expressed but does not activate transcription of Minos transposase unless Doxycycline is administered to the animal.

Alternative inducible systems include or tamoxifen inducible transposase (a modified oestrogen receptor domain (Indra et al., (1999) Nucl. Acid Res. 27:4324-27) coupled to the transposase which retains it in the cytoplasm until tamoxifen is given to the culture), or a RU418 inducible transposase (operating under the same principle with the glucocorticoid receptor; see Tsujita et al., (1999) J. Neuroscience 19:10318-23).

In addition, any of these promoters may be modified by the addition of further regulatory sequences, for example enhancer sequences. Chimeric promoters may also be used comprising sequence elements from two or more different promoters described above.

The use of locus control regions (LCRs) is particularly preferred. LCRs are capable of conferring tightly-regulated tissue specific control on transgenes, and to greatly increase the fidelity of transgene expression. A number of LCRs are known in the art. These include the β-globin LCR (Grosveld et al., (1987) Cell 51:975-985); α-globin (Hatton et al., (1990) Blood 76:221-227; and CD2 (Festenstein et al., (1996) Science 271:1123-1125), plus immunoglobulins, muscle tissue, and the like.

Regulation of transposase and/or transposon expression may also be achieved through the use of ES cells. Using transformed ES cells to construct chimeric embryos, it is possible to produce transgenic organisms which contain the transposase genes or transposon element in only certain of their tissues. This can provide a further level of regulation.

The regulation of expression of transposase may induce excision of a transposon. This may be used to genetically manipulate an organism. As used herein, the term “genetically manipulate” refers to the manipulation of genes in an organism's genome and may include the insertion or excision of a gene or part of a gene.

The sequence of the transposase may be modified to optimise codon usage and thus, increase transposition frequencies. “Codon usage” refers to the frequency pattern in which a given organism uses the 64 possible 3 letter codons of the genetic code in its coding sequences. Because of codon usage preferences, transgenes exhibiting a codon usage pattern more similar to that of the transgenic host organism will generally be more efficiently expressed than those exhibiting a widely differing codon usage pattern. Optimisation of codon usage by converting less frequently used codons to more frequently used codons is a method well known in the art to increase the expression levels of a given gene. Information on codon usage is widely known for a broad range of species (see, e.g., “Codon Usage Tabulated From The International DNA Sequence Databases Status For The Year 2000,” Nakamura et al., Nucl. Acids Res. 28, 292). Codon usage is considered “optimized” when at least one codon in the transposase coding region is replaced with a codon that is used more frequently (i.e., at least 1% more frequently, but preferably at least 5%, 10%, 15%, 20% or more) in the transgenic host species than that encoded by the species from which the transposase is originally taken.

The invention is further described, for the purpose of illustration, in the following examples.

### EXAMPLES

### Plasmid Constructions

The helper plasmid CD2/ILMi is constructed by subcloning the transposase cDNA (Klinakis et al., (2000) EMBO reports 1:16-421) as an XbaI-blunt fragment into the vector SVA(−). The SVA(−) vector is a derivative of the VA vector (Zhumabekov et al., (1995) J. Immunol. Methods 185:133-140) with extended multiple cloning sites.

Transposon MiCMVGFP is constructed as follows: The plasmid pMILRTetR (Klinakis et al., (2000) Ins. Mol. Biol. 9:269-275 (2000b) is cut with BamHI and re-ligated to remove the tetracycline resistance gene between the Minos ends, resulting in plasmid pMILRΔBamH1. An Asp718/SacI fragment from pMILRΔBamH1, containing the Minos inverted repeats and original flanking sequences from D. hydei, is cloned into plasmid pPolyIII-I-lox (created by insertion of the loxP oligo:

ATAACTTCGTATAGCATACATTATACGAAGTTAT

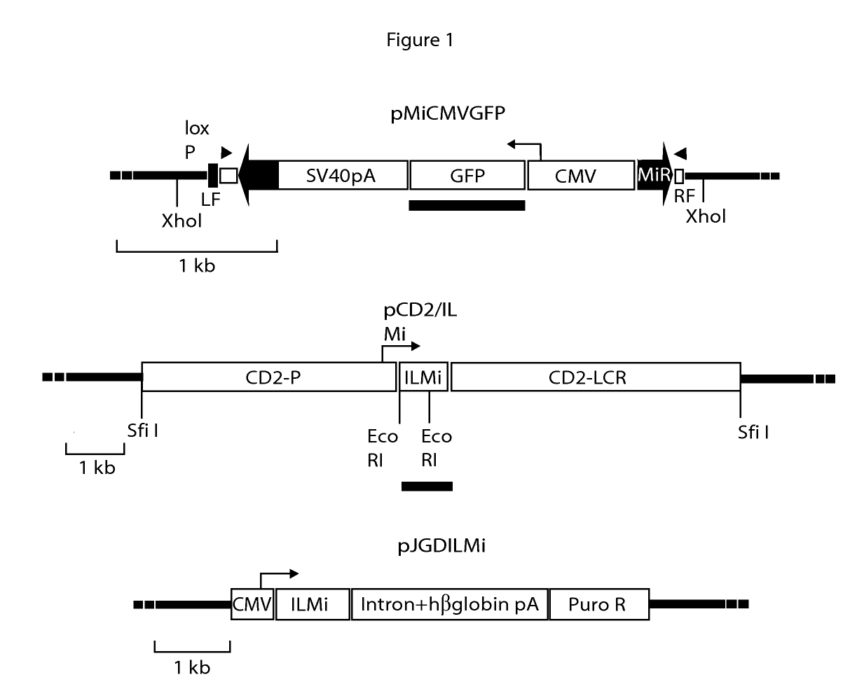

into the Asp718 site of the vector pPolyIII-I (accession No. M18131), resulting in plasmid ppolyMILRΔBamH. The final construct (pMiCMVGFP, FIG. 1) used for the generation of transgenic mice, is created by inserting into the Spe I site of ppolyMILRΔBamH1 the 2.2 kb SpeI fragment from plasmid pBluescriptGFP, containing a humanised GFP gene (from Clontech plasmid pHGFP-S65T) driven by the CMV promoter and followed by the SV40 intervening sequence and polyadenylation signal.

Plasmid pJGD/ILMi (FIG. 1) is constructed as follows: A 1 kb EcoRV/NotI fragment containing the Minos transposase cDNA is cloned into EcoRV/NotI of plasmid pJG-3 (the puro variant of pJG-1; Drabek et al., (1997) Gene Ther. 4:93-100. The resulting plasmid (pJGD/transposase) that carries a CMV promoter upstream of the transposase cDNA, an intron with splice site and polyA from the human β globin gene and the puromycin resistance gene driven by PGK promoter and followed by the poly(A) signal from the bovine growth hormone gene is used as the transposase source in transfections of embryonic fibroblasts.

### Generation of Transgenic Mice

The transposase-expressing TM2 mouse line is generated by injecting the 12.5 kb SfiI fragment from the CD2/ILMi plasmid (FIG. 1) into CBA×C57 B1/10 fertilized oocytes. Transgenic founder animals are identified by Southern blotting of DNA from tail biopsies, using the 1 kb transposase cDNA fragment as a probe and crossed with F1 CBA×C57 B1/10 mice to generate lines.

The transposon-carrying MCG line is constructed by injecting the 3.2 kb XhoI fragment from the pMiCMVGFP plasmid into FVB×FVB fertilized oocytes. Transgenic founder animals are identified by Southern blotting of DNA from tail biopsies, using GFP DNA as a probe.

Cell Culture, Transfection

13.5 day pregnant females (from crosses between MCG heterozygous transgenic male and wt females) are sacrificed, embryos are isolated and part of the material is used for genotyping. The remaining embryonic tissue is minced using a pair of scissors and immersed in a thin layer of F10/DMEM culture medium supplemented with 10% FCS and antibiotics. Two spontaneously immortalized mouse embryonic fibroblasts lines (MEFs) with MCG/+genotype are obtained by subculturing of primary MEFs. They are stably transfected with 20 μg of plasmid pJGD/ILMi linearised with ScaI, using Lipofectin (GibcoBRL). Transfectants are selected on puromycin at a concentration of 1 μg/ml.

### Northern Blot Hybridisation

15 μg of total RNA isolated (Chomozynski & Sacchi, (1987) Analytical Biochem. 162:156-159) from kidney, thymus and spleen is subjected to electrophoresis in a 1.2% agarose gel containing 15% formaldehyde. Northern blot analysis is performed as described previously (Sambrook et al., (1989) Molecular Cloning, A Laboratory Manual, Cold Spring Harbor Laboratory Press, Cold Spring Harbor, N.Y.).

### PCR Analysis

Genomic DNA from different tissues is isolated with the DNeasy Tissue-Kit (QIAGEN) according to the manufacturer's instructions. PCR reactions are performed using primers 11DML:

(5′AAGTGTAAGTGCTTGAAATGC-3′)

### and GOUM67:

(5′-GCATCAAATTGAGTTTTGCTC-3′).

PCR conditions are as follows: 10 mM Tris-HCl (pH 8.8), 50 mM KCl, 1.5 mM MgCl₂, 0.001% gelatin; 1.2 units Taq 2000™ DNA Polymerase (STRATAGENE), 200 g template DNA and 10 pmol of each primer per 25 μl A reaction. 43 or 60 cycles of 30″ at 94° C., 30″ at 59° C. and 30″ at 72° C. were performed. PCR products are cloned into the PCRII TA cloning vector (Invitrogen) and are sequenced using the T7 primer.

### DNA Fluorescent In Situ Hybridisation (FISH) Analysis

Cells from minced thymus or spleen are cultured for 48 h in RPMI medium (GIBCO BRL) supplemented with 9% FCS (GIBCO BRL), 13.6% Hybridoma medium (GIBCO BRL), 3.4 μg/ml Lithium chloride (MERCK), 7.2 μg/ml Concanavaline-A (SIGMA), 22.7 i.u./ml Heparine (LEO), 50 μM Mercaptoethanol, 25.4 μg/ml L.P.S. (SIGMA), 10 ng/ml interleukin 6 (PEPROTECH EC LTD). Chromosome preparations and FISH are carried out as described previously (Mulder et al., (1995) Hum. Genet. 96:133-141). The 737 bp SacI/NotI GFP fragment from the pMiCMVGFP construct is used as a probe. The probe is labelled with Biotin (Boehringer Manheim) and immunochemically detected with FITC. A telomeric probe for chromosome 14 (Shi et al., (1997) Genomics 45:42-47) is labelled with dioxygenin (Boehringer Manheim) and immunochemically detected with Texas Red.

### Example 1

### Activation of Minos In Vivo in a Tissue-Specific Manner

Two transgenic mouse lines are generated to determine whether Minos can transpose in mouse tissues: One containing a Minos transposon and another containing the Minos transposase gene expressed in a tissue-specific manner. The transposon-carrying line (line MCG) contains a tandem array of a fragment containing a Minos transposon (MiCMVGFP, FIG. 1) containing the GFP gene under the control of the cytomegalovirus promoter. The transposon is engineered such that almost all sequence internal to the inverted repeats is replaced by the CMV/GFP cassette. Not containing the transposase-encoding gene, this transposon is non-autonomous, and can only be mobilized when a source of transposase is present. The transposase-expressing line (line TM2) contains a tandem array of a construct comprising the Minos transposase cDNA under the control of the human CD2 locus, consisting of the CD2 promoter and LCR elements (pCD2/ILMi, FIG. 1). In transgenic mice, the human CD2 locus is transcribed at high levels in virtually all thymocytes as well as peripheral T cells (Zhumabekov et al., (1995) J. Immunol. Methods 185:133-140).

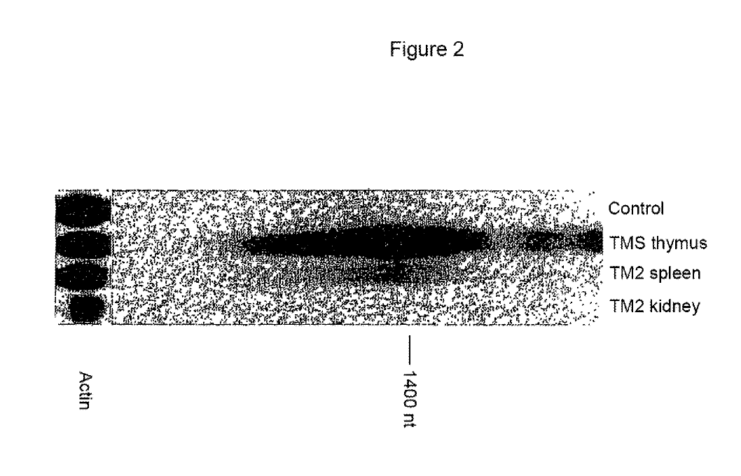

Heterozygous TM2/+ mice are tested for tissue-specific production of Minos transposase RNA by Northern blot analysis. Minos transposase mRNA is detected in thymus and spleen, the two organs with large numbers of T cells, but is not detected in other organs such as kidney (FIG. 2).

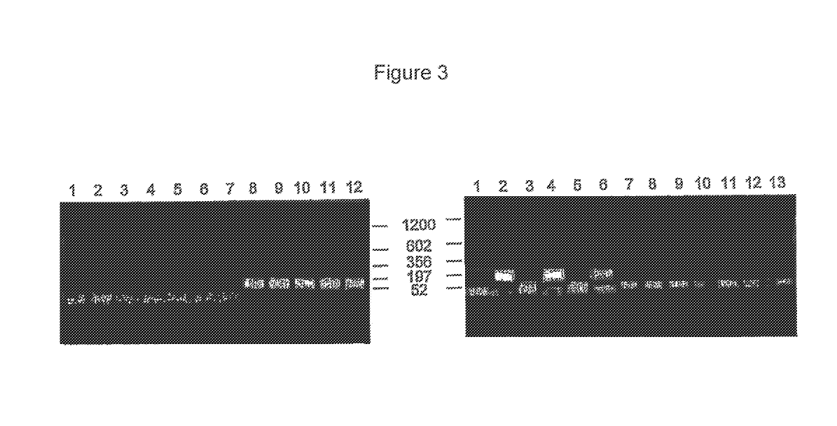

A PCR assay for transposon excision is used to detect active transposition by Minos transposase in mouse tissues, using primers that hybridise to the non-mobile Drosophila hydei sequences which flank the Minos transposon in the constructs shown in FIG. 1 (Klinakisv et al., (2000) Ins. Mol. Biol. 9:269-275). In Drosophila cells, transposase-mediated excision of Minos is followed by repair of the chromatid which usually leaves a characteristic 6-base pair footprint (Arca et al., (1997) Genetics 145:267-279). With the specific pair of primers used in the PCR assay this creates a diagnostic 167 bp PCR fragment (Catteruccia et al., (2000) Proc. Natl. Acad. Sci. U.S.A. 97:2157-2162). As shown in FIG. 3, the diagnostic band is present in tissues of double transgenic (MCG/+TM2/+) mice expressing the transposase, but not of MCG/+ mice, not expressing transposase. The identity of the fragment is confirmed by Southern blot analysis using a labelled DNA probe specific for the amplified sequence (data not shown). Excision is detectable mainly in thymus and spleen of the double transgenics; lower levels of excision are detectable in liver (FIG. 3). Very low levels of excision can also be detected in kidney, brain, and skeletal muscle, after 15 additional cycles of amplification (data not shown). Low levels of expression of the human CD2 locus in liver and lung of transgenic mice has been documented previously (Lang et al., (1988) EMBO J. 6:1675-1682). We therefore attribute the excision detected in tissues other than thymus and spleen to the presence of small numbers of T cells or to the expression of transposase in non-T cells of these tissues due to position effects.

### Example 2

### Detection of Transposition in Cultured Embryonic Fibroblasts

The PCR excision assay is used to detect Minos excision in cultured embryonic fibroblasts carrying the MCG transgene. Cells are transfected with a plasmid carrying the Minos transposase cDNA under CMV control (pJGD/ILMi, FIG. 1) and analysed by the PCR excision assay. Excision products are detectable in transfected but not in non-transfected cells (data not shown). This result suggests that the transposon transgene is accessible to the Minos transposase in tissues other than T cells.

### Example 3

### Detection of Excision Events

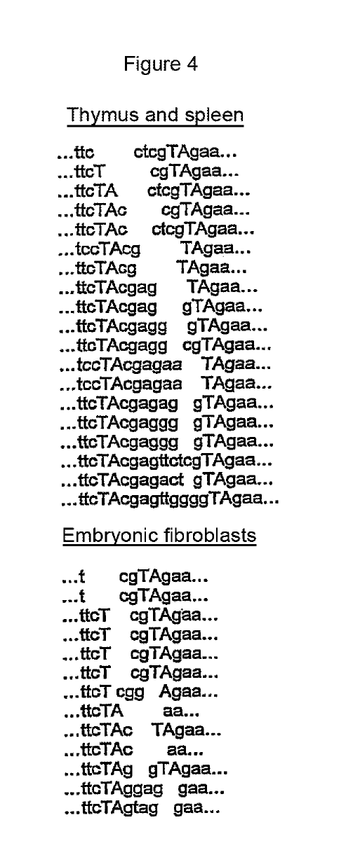

To determine the nature of the excision events, PCR products from thymus and spleen of MCG/+TM2/+ mice and from pJGD/ILMi transfected embryonic fibroblasts are cloned and sequenced. The sequence left behind after Minos excision in Drosophila consists of the TA dinucleotide duplication that is created upon Minos insertion, flanking the terminal 4 nucleotides of the transposon (i.e. either a AcgagT or a ActcgT insertion in the TA target site). In the mouse excisions analysed, the size and sequence of the footprints varies considerably (FIG. 4). Only 2 of the 32 footprints have the typical 6 bp sequence; the others contain extra nucleotides, in addition to complete or partial versions of the typical footprint. Four events have 1-2 nucleotides of the flanking D. hydei chromosomal sequence deleted. The differences in footprint structures observed between Drosophila and mouse may reflect the involvement of host factors in Minos excision and/or chromatid repair following excision.

### Example 4

### Detection of Transposition in Transgenic Mice Using FISH

Detection of transposase-dependent excision in thymus and spleen suggests that transposition may also take place in these tissues. The detection of transposition events is not straightforward, because every transposition event is unique, and as a result the tissue in which transposition has occurred will be a mosaic of cells with unique transpositions. Indeed, Southern analysis did not show transposition events in the thymus of double transgenics, indicating that, if such mosaics exists, they consist of small numbers of clonally related cells.

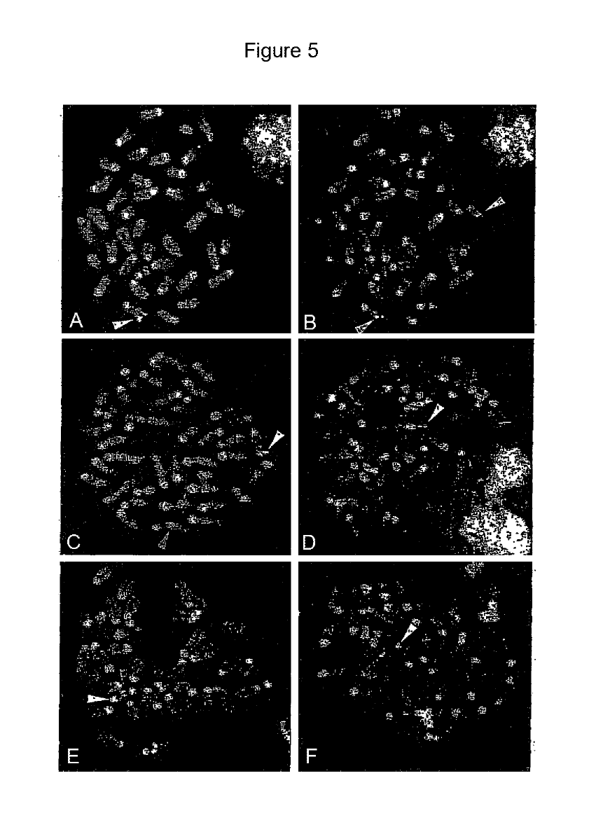

Therefore, FISH in metaphase nuclei from the thymus and spleen to detect individual transposition events. A GFP fragment is used as a probe to detect relocalisation of transposons into new chromosomal positions. The initial position of the array of transposons is at the tip of chromosome 14, at a position indistinguishable from the telomere, as shown by co-localization, in metaphase and interphase chromosomes, with a probe specific for telomeric sequences of chromosome 14 (FIG. 5, A-B). A total of 3,114 metaphases from 5 MCG/+TM2/+ mice are analysed; 1,688 are from spleen and 1,426 from thymus. Nineteen of these metaphases (11 from spleen and 8 from thymus) show transposition. In addition to the signal at the tip of chromosome 14, pairs of dots are present in these metaphases on chromosomes other than 14, or on a new position on chromosome 14. Representative metaphases are shown in FIG. 5 (C-F). Morphological analysis of the chromosomes carrying new insertions show that all events except one are independent from each other, i.e. they represent different transpositions. Analysis of the positive metaphases with a probe specific for the telomere of chromosome 14 indicates that transpositions do not involve translocation of telomeric material (data not shown). As controls, 2,440 metaphases from thymus and spleens of five MCG/+ mice are screened; no transpositions are detectable in those samples.

This is the first demonstration that a transposase expressed from a transgene can mobilize a transposon to jump into new chromosomal sites in mammalian tissues.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for generating, detecting and characterizing one or more genetic mutations in a transgenic mammal, comprising the steps of:
(a) generating a transgenic mammal by:
(i) providing a first transgenic mammal, which mammal comprises, within its genome, one or more copies of a transposon,
(ii) providing a second transgenic mammal, which mammal comprises, in its genome one or more copies of a gene encoding a transposase cognate for said transposon, wherein said transposase is expressed under the control of control sequences which permit regulation of the expression of said transposase; and
(iii) crossing the first transgenic mammal with the second transgenic mammal so as to obtain a transgenic mammal which comprises, in at least a portion of its tissues or cells, both the transposon and the transposase genes wherein expression of said transposase gene is regulated in a tissue specific manner; and
wherein the regulation of expression of the transposase induces excision of one or more transposon from a first position in the genome and insertion of the transposon(s) into a other position(s) in the genome in said cells or tissue;
(b) characterising a change in phenotype of the transgenic mammal produced in step (iii), as compared to either said first or second transgenic mammal; and
(c) detecting the position of one or more transposon insertion events in the genome of the mammal produced in step (iii) or performing sequence analysis to identify the site of insertion in the genome of the mammal produced in step (iii),
wherein steps (b) and (c) are performed in any order.

2. The method of claim 1, further comprising step (d) of correlating the position of the insertion events with the observed phenotype, the position of the insertion events being indicative of the location of one or more gene loci connected with the observed phenotype, whereby said genetic mutation is detected and characterized.

3. The method of claim 2, further comprising step (e) comprising: cloning the genetic loci comprising the insertions, whereby a gene which is correlated with a phenotypic characteristic is isolated and identified

4. The method of claim 2 or 3, wherein a regulatory element is isolated and identified.

5. The method of claim 4 wherein the regulatory element is an enhancer

6. The method of claim 1 or 2 wherein the insertion event results in a tumour

7. The method of claim 3 wherein the identified gene is an oncogene

8. The method of claim 4 wherein the regulatory element modulates tumour formation
